(19) United States
(12) Patent Application Publication
(10) Pub. No.: US 2025/0302395 A1
(43) Pub. Date: Oct. 2, 2025

(54) METHOD AND SYSTEM FOR DETERMINING PREDICTIVE INDEX INDICATING LEVEL TO WHICH HUMAN SUBJECT IS AT RISK OF DEVELOPING HYPOACTIVE DELIRIUM

(71) Applicant: LG ELECTRONICS INC., Seoul (KR)

(72) Inventors: Davide DI CENSO, Oakland, CA (US); Joshua MILBURN, Roanoke, VA (US); Philip BELL, New York, NY (US)

(73) Assignee: LG ELECTRONICS INC., Seoul (KR)

(21) Appl. No.: 19/091,558

(22) Filed: Mar. 26, 2025

Related U.S. Application Data
(60) Provisional application No. 63/571,390, filed on Mar. 28, 2024.

(51) Int. Cl. A61B 5/00; A61B 5/11; G16H 50/30; G16H 50/70

(52) U.S. Cl. CPC A61B 5/7275; A61B 5/002; A61B 5/1128; A61B 5/4076; A61B 5/4803; A61B 5/4806; A61B 5/7264; A61B 5/746; G16H 50/30; G16H 50/70

(57) ABSTRACT

According to at least one embodiment, a method of determining a predictive index indicating a level to which a human subject is at risk of developing hypoactive delirium includes: extracting first features of first audio content and first video content continuously capturing the human subject in a setting over a first period; establishing a behavioral baseline specific to the human subject based on the extracted first features; providing the established behavioral baseline to a neural network; extracting second features of second audio content and second video content continuously capturing the human subject in the setting over a second period subsequent to the first period; providing the extracted second features to the neural network for determining the predictive index based on the established behavioral baseline and the extracted second features; and outputting an alert based on the determined predictive index being above a threshold value.

### CROSS-REFERENCE TO RELATED APPLICATION(S)

Pursuant to 35 U.S.C. § 119 (e), this application claims the benefit of U.S. Provisional Patent Application No. 63/571,390, filed Mar. 28, 2024, the contents of which are hereby incorporated by reference herein in its entirety.

### BACKGROUND

Delirium is a state of acute confusion, in which symptoms may include disturbances in attention, awareness, and higher-order cognition. One type of delirium is hypoactive delirium. Hypoactive delirium can cause subtle changes such as unusual drowsiness and lethargy. A person experiencing hypoactive delirium may not respond to caregivers or family members, or may seem dazed in general. Such a person may seem withdrawn, sluggish or tired, or unusually sleepy. The person may interact less with people around him or her, struggle to stay focused when awake, and eat or drink less than usual.

Hypoactive delirium poses extremely high costs to healthcare institutions. For example, delirium may complicate hospital stays for 20% of the 11.8 million persons aged 65 and older who are hospitalized each year. In addition, hypoactive delirium may account for over $143 billion in costs nationally.

Identifying hypoactive delirium in its earliest stages (including situations in which a person does not exhibit a severe presentation of existing hypoactive delirium, but is at risk of developing hypoactive delirium) can greatly improve clinical outcomes and mitigate downstream impacts on both patient outcomes and costs incurred. For example, identifying this affliction earlier rather than later may preempt the occurrence of significant adverse consequences in patient outcomes—e.g., falls, pressure sores, increased length of hospital stay, and increased risk of declining health and death.

However, hypoactive delirium may be difficult to recognize early. For example, as noted earlier, hypoactive delirium can cause subtle changes such as unusual drowsiness and lethargy. For this reason, a person who appears tired or sleepy due to early stages of hypoactive delirium may be misdiagnosed as someone experiencing depression or dementia, or merely as one who is simply tired or sleepy. In addition, anomalies that are indicative of delirium risk may be too subtle to be recognized by a human observer (e.g., too subtle to be recognized by the human eye).

### SUMMARY

One approach of predicting delirium in critically ill older persons involves using electroencephalogram (EEG) machines that record electrical activity of the brain. However, use of such machines is invasive, and involve a high degree of contact with respect to the person being monitored. In addition, use of such machines typically requires human (e.g., manual) observation.

In severe presentations of pre-existing hypoactive delirium, one or more of the following features may be present: unawareness; decreased alertness; sparse/slow speech; lethargy; slowed movements; staring; and/or apathy. In situations where such features are present to degrees that are recognizable to the human eye, these features may be useful in supporting a finding of pre-existing hypoactive delirium. However, such situations may not be helpful when attempting to identify anomalies that are indicative of delirium risk.

Aspects of this disclosure are directed towards identifying such anomalies that would most likely go unnoticed by a human observer such as a human clinician. The anomalies are identified using information recorded by non-contact sources (e.g., sources excluding an EEG machine). Such information may include multimodal audio and video data collected at a setting in which a human subject is located (e.g., a room in which a patient is located). Aspects of this disclosure are directed not merely to detecting severe presentations of pre-existing hypoactive delirium, but rather to identifying anomalies that are indicative of delirium risk and/or predicting whether the human subject is at risk of developing hypoactive delirium.

Aspects of this disclosure are directed toward improving detection of hypoactive delirium in an early (or earliest) stages. According to one or more aspects, a camera-based system is used to continuously monitor a human subject in order to detect potential symptoms of hypoactive delirium. For example, the monitoring of the subject may occur in an inpatient hospital setting or a long-term care (LTC) facility. According to one or more aspects, the system may use a multi-modal model that analyzes video and/or audio data over time to detect early warning signs of hypoactive delirium.

According to at least one embodiment, a method of determining a predictive index indicating a level to which a human subject is at risk of developing hypoactive delirium is disclosed. The method includes: extracting first features of first audio content and first video content continuously capturing the human subject in a setting over a first period, the extracted first features detailing a plurality of first behavioral aspects of the human subject over the first period, wherein at least one of the plurality of first behavioral aspects is detailed at a granularity that is undetectable by a human observer; establishing a behavioral baseline specific to the human subject based on the extracted first features; providing the established behavioral baseline to a neural network; extracting second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer; providing the extracted second features to the neural network for determining the predictive index indicating the level to which the human subject is at risk of developing hypoactive delirium, based on the established behavioral baseline and the extracted second features; and outputting an alert based on the determined predictive index being above a threshold value.

According to another embodiment, an artificial intelligence (AI) device configured to determine a predictive index indicating a level to which a human subject is at risk of developing hypoactive delirium is disclosed. The AI device includes: at least one transceiver; and at least one processor. The at least one processor is configured to: extract first features of first audio content and first video content continuously capturing the human subject in a setting over a first period, the extracted first features detailing a plurality of first behavioral aspects of the human subject over the first period, wherein at least one of the plurality of first behavioral aspects is detailed at a granularity that is undetectable by a human observer; establish a behavioral baseline specific to the human subject based on the extracted first features; provide the established behavioral baseline to a neural network; extract second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer; provide the extracted second features to the neural network for determining the predictive index indicating the level to which the human subject is at risk of developing hypoactive delirium, based on the established behavioral baseline and the extracted second features; and output an alert based on the determined predictive index being above a threshold value.

According to another embodiment, a non-transitory storage medium store instructions that, when executed, cause at least one processor to perform operations. The operations include: extracting first features of first audio content and first video content continuously capturing a human subject in a setting over a first period, the extracted first features detailing a plurality of first behavioral aspects of the human subject over the first period, wherein at least one of the plurality of first behavioral aspects is detailed at a granularity that is undetectable by a human observer; establishing a behavioral baseline specific to the human subject based on the extracted first features; providing the established behavioral baseline to a neural network; extracting second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer; providing the extracted second features to the neural network for determining a predictive index indicating a level to which the human subject is at risk of developing hypoactive delirium, based on the established behavioral baseline and the extracted second features; and outputting an alert based on the determined predictive index being above a threshold value.

### BRIEF DESCRIPTION OF THE DRAWINGS

FIG. 1 illustrates an example monitoring of a human subject (e.g., a person) according to at least one embodiment.

FIG. 2 illustrates a block diagram of a system according to at least one embodiment.

FIG. 3 illustrates a flowchart of a method of determining a predictive index indicating a level to which a human subject is at risk of developing hypoactive delirium.

FIG. 4 is a block diagram of an artificial intelligence (AI) device according to at least one embodiment.

FIG. 5 is a diagram illustrating a system in which an AI device and devices operable by one or more PSM professionals are linked according to at least one embodiment.

### DETAILED DESCRIPTION

Hereinafter, embodiments disclosed in the present specification will be described in detail with reference to the accompanying drawings, the same or similar elements regardless of a reference numeral are denoted by the same reference numeral, and a duplicate description thereof will be omitted. In the following description, the terms “module” and “unit” for referring to elements are assigned and used exchangeably in consideration of convenience of explanation, and thus, the terms per se do not necessarily have different meanings or functions. Wherever possible, the same reference numbers will be used throughout the drawings to refer to the same or like parts. In the following description, known functions or structures, which may confuse the substance of the present disclosure, are not explained. The accompanying drawings are used to help easily explain various technical features, and it should be understood that the embodiments presented herein are not limited by the accompanying drawings. As such, the present disclosure should be construed to extend to any alterations, equivalents, and substitutes in addition to those which are particularly set out in the accompanying drawings.

Terminology used herein is used for the purpose of describing particular example implementations only and is not intended to be limiting. As used herein, the singular forms “a,” “an,” and “the” may be intended to include the plural forms as well, unless the context clearly indicates otherwise. The terms “comprises,” “comprising,” “includes,” “including,” “containing,” “has,” “having” or other variations thereof are inclusive and therefore specify the presence of stated features, integers, steps, operations, elements, and/or components, but do not preclude the presence or addition of one or more other features, integers, steps, operations, elements, components, and/or groups thereof. Furthermore, terms such as “first,” “second,” and other numerical terms, are used only to distinguish one element from another element. These terms are generally only used to distinguish one element from another.

Hereinafter, implementations of the present disclosure will be described in detail with reference to the accompanying drawings. Like reference numerals designate like elements throughout the specification, and overlapping descriptions of the elements will not be provided. When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present.

In hospitals, signs of delirium are often identified through manual observation. Signs of hypoactive delirium are often subtle. For example, delirium may be characterized by acute and fluctuating changes in attention, cognition, and awareness. Therefore, it may be important for clinicians to monitor any significant or subtle change relative to normal behavior for each individual patient.

Clinical signs of delirium onset may include disturbances in attention, such as difficulty in focusing, or in sustaining or shifting attention. Afflicted patients may exhibit disorganized thinking, slowed speech, incoherent speech, and impaired memory. Delirium can present itself with physical decline, such as subtle fidgeting, decreased mobility, or other distinct changes in mobility. Fluctuations in alertness and awareness may be common, with individuals experiencing periods of hyperactivity (e.g., a state of heightened psychophysiological arousal) followed by hypoactivity (e.g., a state between normal wakefulness/alertness and coma). Other clinical manifestations may include perceptual disturbances, such as hallucinations or illusions, and disturbances in sleep-wake cycles.

In general medical units, which make up the majority of many hospitals, a physician typically sees a patient in-person once a day, at which point the patient is observed for concerning signs—e.g., concerning signs of hypoactive delirium. Nurses who check on patients approximately once every 2 to 6 hours may also observe the patient and alert physicians to concerning signs of hypoactive delirium.

However, such clinician visits and spot checks typically occur infrequently. The frequency at which such clinician visits and spot checks occur at the patient's bedside may not be sufficiently high to detect hypoactive delirium in its early stages.

Also, signs of hypoactive delirium are often subtle and may fluctuate throughout the day. Such signs may be too subtle to be recognized by the human eye. Also, when the number of spot checks (not only by doctors, but also by nurses) are limited, the likelihood that such signs are observed may be reduced. Also, due to the fluctuating nature of symptoms throughout the day, it is possible that spot checks occur at times of the day when signs of hypoactivity delirium are less noticeable or less visible.

Aspects of this disclosure are directed towards identifying anomalies that are indicative of delirium risk (e.g., anomalies that would most likely go unnoticed by a human observer such as a human clinician). In various aspects, such identification is performed using data recorded by non-contact sources. The non-contact sources do not include devices that require a high degree of physical contact with the patient, such as an EEG machine. The data may include multimodal audio and video data collected at a room in which a patient is located. Aspects of this disclosure are directed not merely to detecting severe presentations of pre-existing hypoactive delirium, but rather to identifying anomalies that are indicative of delirium risk and/or predicting whether a person is at risk of developing hypoactive delirium.

Aspects of this disclosure are directed toward improving detection of hypoactive delirium in an early or earliest stage(s). According to one or more aspects, a camera-based system employing an artificial intelligence (AI) model is used to detect early onset of delirium and/or predict whether a person is at risk of developing hypoactive delirium. According to various embodiments, a camera-based monitoring model is utilized to better analyze subtle changes in a patient continuously (e.g., 24 hours a day, 7 days per week) throughout the patient's hospital stay.

Unlike medical staff members who rotate out every shift, such a camera-based system can monitor behavioral patterns over time more readily, thereby building out continuous context for each patient. With nursing staff at hospitals often stretched thin due to a combination of staffing shortages and the existence of an increasingly sick patient population, supporting or supplementing manual spot-checking with technology-based solutions would provide significant value to hospital or long-term care (LTC) systems.

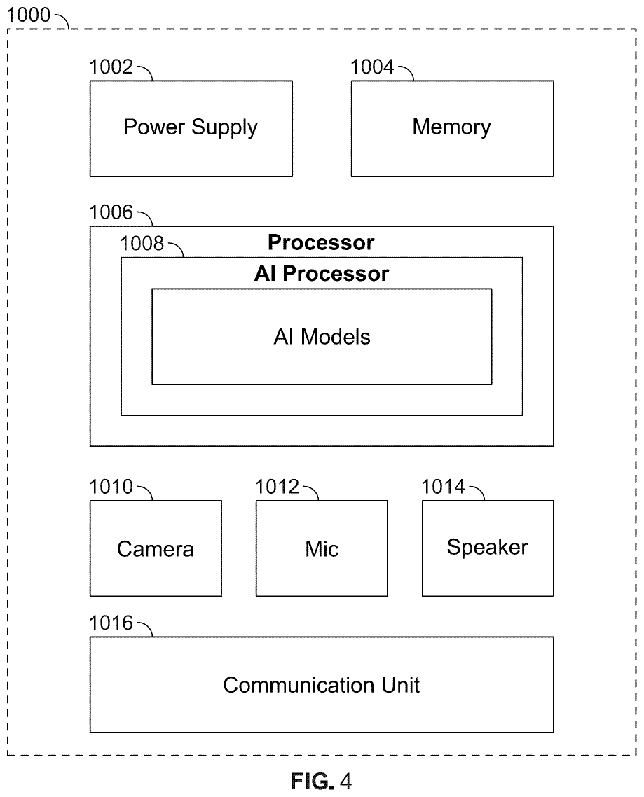

According to various embodiments, a camera including a microphone (or a system or device including such a camera-see, e.g., AI device 1000 of FIG. 4) is provided in a room occupied by a patient in an inpatient unit or LTC facility. As such, the patient's movement and speech patterns can be monitored continuously (e.g., 24 hours a day, 7 days per week).

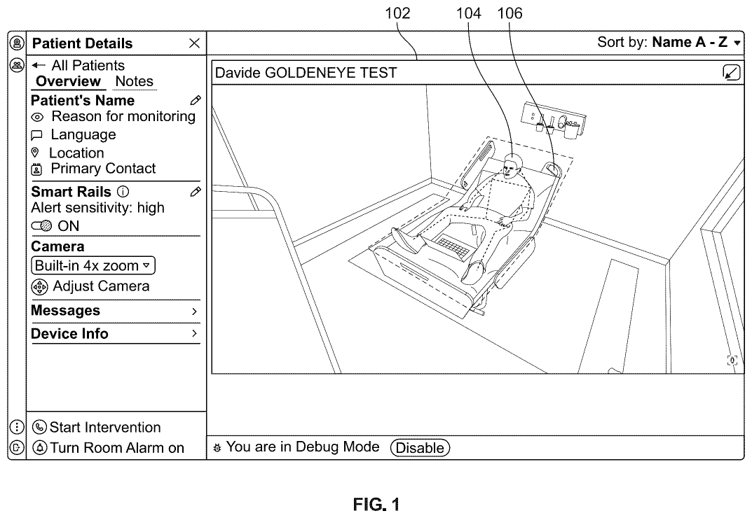

FIG. 1 illustrates an example monitoring of a person according to at least one embodiment. With reference to FIG. 1, display of a video image 102 is provided. The display may be provided at a display device (e.g., a video monitor) that is utilized by one or more patient safety monitoring (PSM) professionals. The video image 102 may be captured by one or more cameras that are positioned in a healthcare setting such as a hospital, a nursing facility, etc. For example, the camera(s) may be positioned in the room of a medical patient. In the example of FIG. 1, the video image 102 captures a human patient 104 who is positioned on a bed 106.

It is understood that the corresponding video may include audio that is captured by the camera(s). For example, one or more microphones (included in or otherwise coupled to the camera(s)) may capture audio sounds made by the human patient 104—e.g., speech sounds, vocal utterances, etc. Captured video content, in combination with corresponding audio content, will be referred to herein as audiovisual content.

Occurrence of events that are captured in the audiovisual content may be detected. Such events may include purely visual events, purely audio events and events having both visual and audio characteristics.

For example, purely visual events may relate to mobility of the human patient 104, including specific movements of the human patient. Such specific movements may include relatively small movements at an extremity of the human patient 104—e.g., fidgeting at or fidgeting gestures made by a hand of the human patient.

As another example, purely visual events may relate to larger movements (or lack thereof) made by the human patient 104, e.g., by the entire body (or a larger portion of the body) of the human patient. In addition, aspects of such movements may be detected. For example, the speed of such movements may be detected, e.g., as a potential sign of decreased mobility.

Detection of such movement (or lack thereof) may be used to detect whether the human patient 104 is asleep. For example, when minimal changes in body positioning are detected while the human patient 104 is in a sleeping position, it may be concluded that the human patient is asleep. Such conclusions may be used to identify normal sleep patterns and deviations therefrom (e.g., disturbances in sleep-wake cycles).

Regarding purely audio events, examples of such events may relate to speech uttered by the human patient. In addition, aspects of such speech may be detected. For example, the speed at which individual vocal sounds (e.g., syllables) are uttered may be detected. A decrease in the speed at which such individual sounds are made may be detected as a sign of slowed speech.

As another example, the level at which such speech is coherent may also be detected. Here, natural language processing (NLP) and/or large language model (LLM) techniques may be used to determine the level at which the speech is coherent or intelligible. Such techniques may also be used to analyze whether the speech represents a level of disorganized thoughts and/or thinking.

It is understood that various other types of events or aspects may be detected. Examples of such events or aspects may be found in the Glasgow Coma Scale, which is used to assess the depth and duration of impaired consciousness and coma, and/or the Bush-Francis Catatonia Rating Scale, which is used to assess catatonia severity and screen for catatonia in psychiatric and neurologic conditions.

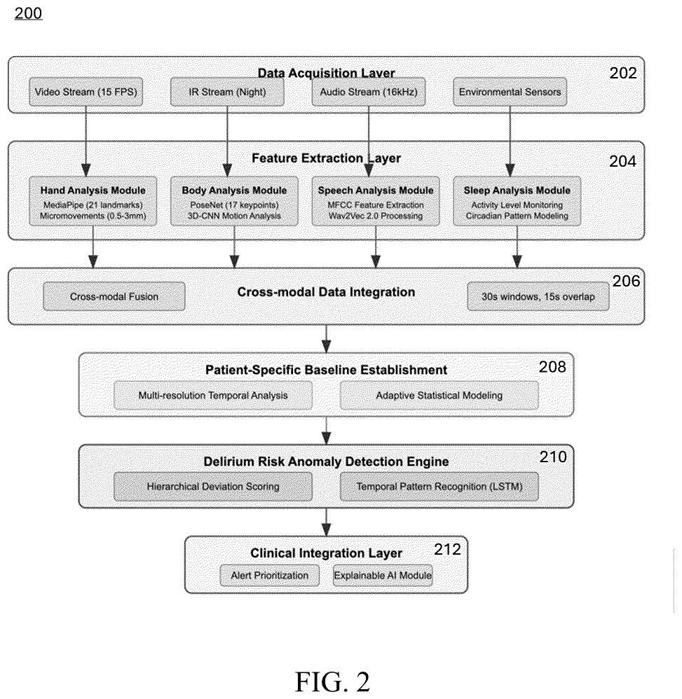

Events that that are captured in the audiovisual content will be described in more detail with reference to FIG. 2.

FIG. 2 illustrates a block diagram of a system 200 according to at least one embodiment.

The system 200 includes a data acquisition layer 202. The data acquisition layer 202 may include data sources such as: one or more sources (or sensors) that acquire a video stream (e.g., one or more day-vision cameras); one or more sources that acquire an infrared (IR) video stream (e.g., one or more night-vision cameras); and one or more sources that acquire an audio stream (e.g., one or more microphones). The microphone(s) may capture audio in windowed samples that overlap one another (e.g., 5-second overlapping windowed samples). The data acquisition layer 202 may include one or more other sources (e.g., environmental sensors). According to various embodiments, the data sources of the data acquisition layer 202 acquire data without requiring contacting of the patient (e.g., physical touching of the patient).

The system 200 further includes a feature extraction layer 204. The feature extraction layer 204 includes multiple modules that operate based on data output by the data acquisition layer 202.

Based on video stream data, a hand analysis module of the feature extraction layer 204 extracts hand landmark positions (keypoints) from the video stream. As will be explained in more detail later, such extracted positions may be used to determine a value of hand position jitter and/or a level of micro-movements of the patient, and, accordingly, identify signs of abnormal hand behavior. According to one or more embodiments, the hand analysis module uses MediaPipe Hands to extract the hand landmark positions.

Also based on video stream data, a body analysis module of the feature extraction layer 204 extracts keypoints for major body joints from the video stream. Such extracted keypoints may be used to determine an activity level and/or a speed of movement of the patient. For example, the activity level may be determined using a sum of the Euclidean distances (e.g., x-y coordinates of the keypoints may be used) between corresponding keypoints in consecutive video frames, averaged over a certain time window with overlapping segments. Here, the time window may be a 24-hour window. Similarly, such distances between corresponding keypoints in particular frames may be used to determine speed of movement. As will be described in more detail later, the body analysis module may identify signs of abnormal body movement. According to one or more embodiments, the body analysis module uses PoseNet to extract the keypoints for major body joints.

Based on audio stream data, a speech analysis module of the feature extraction layer 204 extracts audio features from the audio stream. Such features may include speech rate, pause frequency and duration, and spectral features. For example, the speech rate may be calculated over a particular length (e.g., 30 seconds) using syllable counting. The pause frequency and duration may correspond to a number and an average length of pauses in speech. Here, a pause timestamp annotation technique such as WavBERT may be used. Spectral features may include changes in Mel-Frequency Cepstral Coefficients (MFCCs), spectral centroid, and spectral roll-off. Accordingly, the speech analysis module is capable of detecting subtle speech changes.

Based on video stream data and/or audio stream data, a sleep analysis module of the feature extraction layer 204 detects active/inactive and awake/asleep states of the patient. Regarding the active/inactive state, the sleep analysis module may detect periods of inactivity. For example, PoseNet may be used. If total summed movement across all keypoints (e.g., using Euclidean distance to calculate movement) for a given time window is below a certain number of pixels, then the patient may be considered to be inactive or idle.

Regarding the awake/asleep state, the sleep analysis module may detect whether the eyes of the patient are open or closed. For example, a region of an image around the head of the patient as detected by PoseNet may be provided to software that determines whether the eyes of the patient are open or closed. If the patient's eye are open, then the sleep analysis module may determine that the patient is not asleep.

The system 200 further includes a baseline establishment layer 208. The baseline establishment layer 208 operates based on data output by the feature extraction layer 204 (e.g., via the cross-modal data integration layer 206).

The baseline establishment layer 208 establishes a patient-specific baseline based on data (see, e.g., data acquisition layer 202) recorded continuously over a certain period. Such a period may be longer than or equal to 24 hours and shorter than or equal to 48 hours.

Based on data recorded over this period, each module described earlier with reference to the feature extraction layer 204 may construct a corresponding statistical representation of the patient's behavior. According to various embodiments, this representation may be adopted as a “normal” representation of the patient's behavior. Construction of this representation may employ using a weighted moving average, a Kalman filter and/or another appropriate statistical model, applied to the extracted features over time.

By way of example, the representation constructed by the hand analysis module of the feature extraction layer 204 may include a mean or average jitter as a baseline representation of jitter of the hand(s) of the patient. The representation constructed by the hand analysis module may also include a mean or average movement distance of the hand(s) of the patient.

Also by way of example, the representation constructed by the body analysis module of the feature extraction layer 204 may include an mean or average activity level as a baseline representation of the activity level of the patient. The representation constructed by the body analysis module may also include a mean or average speed of movement of the patient.

According to one or more embodiments, the overall baseline established by the baseline establishment layer 208 is a composite of the module representations at a given time (or time point) representing a holistic view of the patient's behavior at or around that time.

The system 200 further includes a delirium risk anomaly detection engine 210. The delirium risk anomaly detection engine 210 operates based on data output by the baseline establishment layer 208, as well as data output by the feature extraction layer 204. As described earlier with reference to the baseline establishment layer 208, data recorded continuously over a certain period (e.g., a period between 24 and 48 hours in duration) was used to establish a patient-specific baseline. At a further period that is subsequent to that baseline period (i.e., a second monitoring period), further data of that patient is recorded continuously, and is then processed by the feature extraction layer 204 to generate outputs similar to those described earlier with respect to establishment of the patient-specific baseline. At the delirium risk anomaly detection engine 210, such further outputs are assessed relative to the patient-specific baseline.

In at least one embodiment, the delirium risk anomaly detection engine 210 analyses the stream of data produced from modules of the feature extraction layer 204, and performs classification and anomaly detection to determine potential delirium precursors.

By way of example, as described earlier, the patient-specific baseline may include a mean or average jitter as a baseline representation of jitter of the hand(s) of the patient. For the second monitoring period, the feature extraction layer 204 also outputs a further value of the jitter of the hand(s) of the patient. According to one or more embodiments, if the further value of the jitter exceeds the baseline value of the jitter by a certain degree (e.g., by two standard deviations), then the delirium risk anomaly detection engine 210 may interpret this occurrence as a potential indicator of delirium risk.

Also by way of example, as described earlier, the patient-specific baseline may include a mean or average movement distance of the hand(s) of the patient. For the second monitoring period, the feature extraction layer 204 also outputs a further value of the movement distance of the hand(s) of the patient. According to one or more embodiments, if pixel displacement of keypoints that is greater than 0 pixels but less than the baseline value of the hand movement distance occurs at a frequency greater than or equal to a particular threshold (e.g., a threshold number of movements per minute), then the delirium risk anomaly detection engine 210 may interpret this occurrence as a potential indicator of delirium risk.

Also by way of example, as described earlier, the patient-specific baseline may include a mean or average activity level of the body of the patient. For the second monitoring period, the feature extraction layer 204 also outputs a further value of the activity level of the body of the patient. According to one or more embodiments, if it is determined that the activity level has decreased by at least a threshold percentage relative to the patient's baseline activity level, then the delirium risk anomaly detection engine 210 may interpret this occurrence as a potential indicator of delirium risk.

As yet another example, as described earlier, the patient-specific baseline may include a mean or average speed of movement of the patient. For the second monitoring period, the feature extraction layer 204 also outputs a further value of the speed of movement of the patient. According to one or more embodiments, if it is determined that the speed of movement has decreased by at least a threshold percentage relative to the patient's baseline speed of movement, then the delirium risk anomaly detection engine 210 may interpret this occurrence as a potential indicator of delirium risk.

The delirium risk anomaly detection engine 210 may integrate the outputs from the modules to generate an overall hypoactive delirium preliminary risk score. The engine 210 may employ a Long Short-Term Memory (LSTM) recurrent neural network with an attention mechanism, or another suitable algorithm for combining time-series data, such as a rule-based system, a weighted average, or a simpler machine learning model (e.g., logistic regression). According to at least one embodiment, the delirium risk anomaly detection engine 210 outputs a single risk score, ranging from 0 to 1, representing the estimated probability of hypoactive delirium.

The system 200 further includes a clinical integration layer 212. If the risk score output by the delirium risk anomaly detection engine 210 exceeds a particular threshold (e.g., 0.7), then the clinical integration layer 212 may trigger a warning for clinical review. The value of this threshold may be determined through experimental analysis with clinicians, to reduce false positives, and may be adjusted by clinicians via a user interface (UI). This enable a user to quickly adjust the responsiveness of the alerting system.

As described earlier with reference to at least one embodiment, the system 200 operates using four modules of the feature extraction layer 204. However, it is understood that a fewer number of modules may be employed. For example, a combination of two or more of such modules may be employed. However, a greater number of modules may result in a higher degree of accuracy.

Also, it is understood that the modules described herein are illustrative, and that other modules not explicitly described herein may also be employed. For example, the system 200 may also employ other modules running on the edge that can generate outputs based on audio and/or video content for predicting delirium risk.

According to at least one embodiment, the delirium risk anomaly detection engine 210 (running on the edge or on the cloud) may analyze a continual stream of data from one or more models (e.g., one or more other models), and make predictions based on anomalous patterns that may require attention, without explicitly programming those states as rules in the system. According to at least one embodiment, the delirium risk anomaly detection engine 210 may leverage deep learning to further tailor the model results to a specific patient. That is, the delirium risk anomaly detection engine 210 learns the “normal” non-delirium patterns for a specific patient over time.

For example, the established behavioral baseline (see baseline establishment layer 208) may be tailored based on features that are extracted after the baseline was established. In this manner, the tailored behavioral baseline and features that are extracted at yet a further time may be used to generate an update of the delirium risk score.

According to at least one further embodiment, the delirium risk anomaly detection engine 210 also considers contextual data about the patient from external sources (e.g., an electronic health record (HER), the patient's medical history). In this manner, prediction capability may be improved. By way of example, such contextual data may include information regarding prior medical conditions, clinical family history, current and past medications (e.g., prescription medications), previously observed early onset signs and pre-conditions, etc.

According to at least one further embodiment, the delirium risk anomaly detection engine 210 also considers information input by a user. For example, a user (e.g., a nurse or other health-care provider) may input a text-based description of the patient's condition via a UI. In this regard, the engine 210 may utilize natural language processing (NLP) models and/or large language models (LLMs) to translate the input text into a meaningful context. Similar to earlier-noted contextual data from external sources, information input by a user may also be used to improve prediction capability.

According to at least one further embodiment, the predictive index may further be based on clinical data of larger populations of human subjects. For example, a baseline “signal” of “normal” speech characteristics may be built using existing, open clinical datasets to determine population average “healthy” patterns. Speech activity of a patient under monitoring may be classified as “abnormal” if it deviates from the patient baseline and classifies more closely to the “unhealthy” samples of the larger baseline “signal” instead of the “healthy” samples of the larger signal.

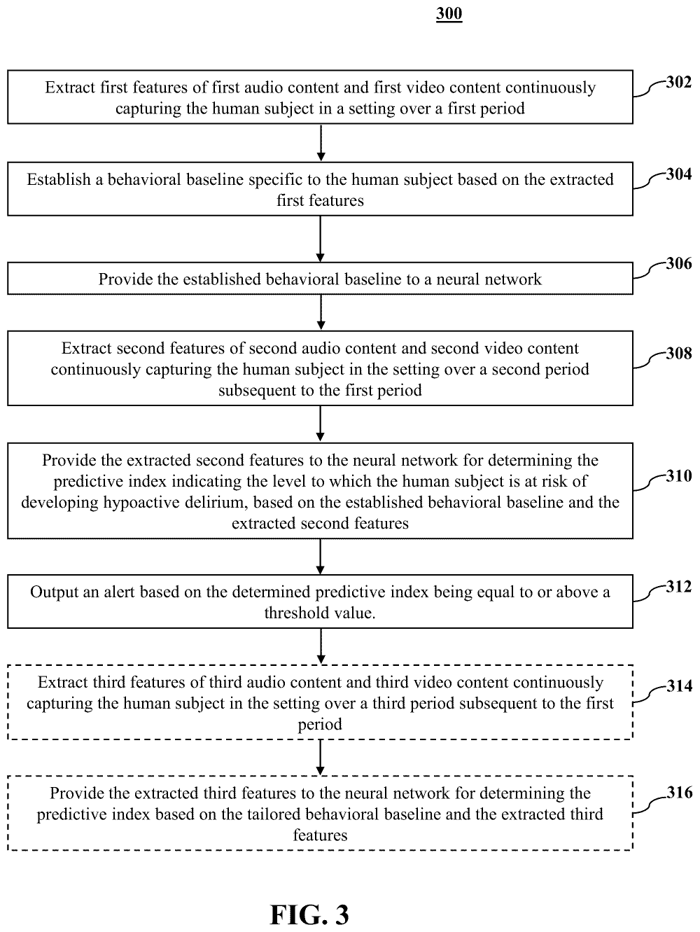

FIG. 3 illustrates a flowchart 300 of a method of determining a predictive index indicating a level to which a human subject is at risk of developing hypoactive delirium.

At block 302, first features of first audio content and first video content continuously capturing the human subject in a setting over a first period are extracted. The extracted first features detail a plurality of first behavioral aspects of the human subject over the first period. At least one of the plurality of first behavioral aspects is detailed at a granularity that is undetectable by a human observer.

For example, with reference back to FIG. 2, modules of the feature extraction layer 204 extract features of audio content and video content continuously capturing the human subject over an initial period.

A duration of the initial period may be in a range of 24 to 48 hours.

The first audio content may include windowed samples that overlap one another. For example, each of the windowed samples may be five seconds in length.

The first video content may include video content recorded by at least one day vision camera as well as video content recorded by at least one night vision camera.

At block 304, a behavioral baseline specific to the human subject is established based on the extracted first features.

For example, with reference back to FIG. 2, the baseline establishment layer 208 establishes a patient-specific behavioral baseline based on features extracted by the feature extraction layer 204.

At block 306, the established behavioral baseline is provided to a neural network (e.g., the delirium risk anomaly detection engine 210 of FIG. 2).

At block 308, second features of second audio content and second video content continuously capturing the human subject in the setting over a second period subsequent to the first period are extracted. The extracted second features detail a plurality of second behavioral aspects of the human subject over the second period. At least one of the plurality of second behavioral aspects is detailed at a granularity that is undetectable by the human observer.

For example, with reference back to FIG. 2, modules of the feature extraction layer 204 extract features of audio content and video content continuously capturing the human subject over a second period subsequent to the initial period.

At block 310, the extracted second features are provided to the neural network (e.g., the delirium risk anomaly detection engine 210 of FIG. 2) for determining the predictive index, based on the established behavioral baseline and the extracted second features.

According to at least one further embodiment, the behavioral baseline is established and the predictive index is determined without using data output by an EEG machine.

According to at least one further embodiment, the plurality of second behavioral aspects of the human subject includes at least one of hand jitter, body movement, speech activity or sleep activity of the human subject.

According to at least one further embodiment in which the plurality of second behavioral aspects of the human subject includes the speech activity of the human subject, the neural network determines the predictive index further based on speech characteristics of larger populations of human subjects. For example, as described earlier with reference to FIG. 2, a baseline “signal” of “normal” speech characteristics may be built using existing, open clinical datasets to determine population average “healthy” patterns. Speech activity of a patient under monitoring may be classified as “abnormal” if it deviates from the patient baseline and classifies more closely to the “unhealthy” samples of the larger baseline “signal” instead of the “healthy” samples of the larger signal.

At block 312, an alert is output based on the determined predictive index being above a threshold value.

For example, with reference back to FIG. 2, the clinical integration layer 212 outputs an alert based on the determined risk score exceeding a particular threshold (e.g., 0.7).

According to at least one further embodiment, at block 314, based on the determined predictive index being less than or equal to the threshold value, the extracted second features are provided to the neural network (e.g., the delirium risk anomaly detection engine 210 of FIG. 2) to train the neural network to tailor the established behavioral baseline based on the extracted second features.

In addition, at block 316, third features of third audio content and third video content continuously capturing the human subject in the setting over a third period subsequent to the second period are extracted. The extracted third features detail a plurality of third behavioral aspects of the human subject over the third period. At least one of the plurality of third behavioral aspects is detailed at a granularity that is undetectable by the human observer. The extracted third features are provided to the neural network for determining the predictive index based on the tailored behavioral baseline and the extracted third features.

For example, with reference back to FIG. 2, the established behavioral baseline (see baseline establishment layer 208) may be tailored based on features that are extracted after the baseline was established. In this manner, the tailored behavioral baseline and features that are extracted at yet a further time may be used to generate an update of the delirium risk score.

As described herein with reference to various embodiments, aspects of this disclosure are directed toward a technologically improved system for the early detection of hypoactive delirium risk. The system employs a non-contact, continuous monitoring approach using multimodal audio and video data acquired from a patient's room. Unlike prior approaches relying on intermittent human observation or invasive, contact-based EEG monitoring, the system leverages an AI model to autonomously establish a patient-specific behavioral baseline and detect subtle deviations indicative of increased delirium risk. The system processes and integrates continuous, high-frequency data streams from multiple sensors, performing complex pattern recognition that is not feasible for human clinicians, thus enabling earlier and more objective identification of at-risk patients. The system performs automated, algorithmic analysis of behavioral patterns, not merely the observation of those patterns.

FIG. 4 is a block diagram of an AI device 1000 (or apparatus) according to at least one embodiment to at least one embodiment.

Referring to FIG. 4, the AI device 1000 is configured to perform features described herein with respect to improving early detection of hypoactive delirium in at least one person located in a healthcare setting (see, e.g., FIG. 3).

The AI device 1000 may include a memory 1004, a processor 1006, and a power supply 1002, and the processor 1006 may further include an AI processor 1008. The processor 1006 may be referred to as a main controller. The AI device 1000 may also include a camera 1010, a microphone 1012, and a speaker 1014. The camera 1010 may capture images including images of the room of a medical patient (see, e.g., video image 102 of FIG. 1).

The AI device 1000 may include an interface 1016. The interface can be configured using at least one of a communication unit (transceiver), a terminal, a pin, a cable, a port, a circuit, an element or a device.

The memory 1004 is electrically connected with the processor 1006. The memory 1004 can store data processed in the processor 1006. With regards to hardware configuration, the memory 1004 may be configured using at least one of a ROM, a RAM, an EPROM, a flash drive, or a hard drive. Also, the memory 1004 may store instructions, that, when executed, cause the processor 1006 to perform features described herein (see, e.g., FIG. 3). The memory 1004 can store various types of data for the overall operation of the AI device 1000, such as a program for processing or control of the processor 1006. The memory 1004 may be integrated with the processor 1006. In one or more particular embodiments, the memory 1004 may be classified as a lower configuration of the processor 1006.

The power supply 1002 can supply power to the AI device 1000. The power supply 1002 can be provided with power from a power source (e.g., a battery) included in the AI device 1000 and can supply the power to each module of the AI device 1000.

The processor 1006 can be electrically connected to the memory 1004, the interface 1016, and the power supply 1002 and exchange signals with these components. The processor 1006 can be realized using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electronic units for executing other functions.

The processor 1006 can be operated by power supplied from the power supply 1002. The processor 1006 can receive data, process the data, generate a signal, and provide the signal while power is supplied thereto by the power supply 1002.

The processor 1006 can receive information from devices connected with the AI device 1000. The processor 1006 can provide control signals to devices connected with the AI device 1000 through the interface 1016.

As described earlier, the processor 1006 may include an AI processor 1008. The AI processor 1008 may operate AI models including one or more analytical models that have been described herein with reference to various embodiments. For example, the one or more analytical models may analyze one or more video images of a video feed, as described earlier with reference to FIG. 1.

The AI device 1000 may include at least one printed circuit board (PCB). The memory 1004, the interface 1016, the power supply 1002, and the processor 1006 may be electrically connected to the PCB.

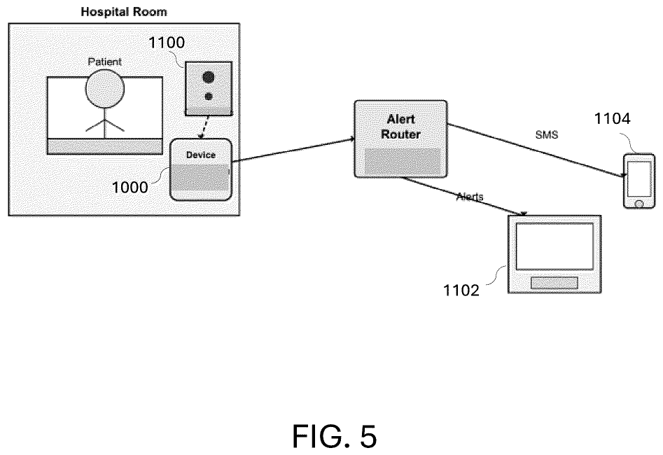

FIG. 5 is a diagram illustrating an AI device (e.g. AI Device 1000 of FIG. 4) and devices operable by one or more PSM professionals are linked according to at least one embodiment. For example, the AI device 1000 is connected to a camera and microphone 1100 in the room, which are configured to monitor the patient continually. In at least one embodiment, the camera and microphone 1100 are integrated together into the AI device 1000, and include standard RGB and night vision cameras. Based on analysis of the features from AI device 1000, an alert may be generated by the AI device 1000, which is then routed to Alert Router (e.g., to a number), which then routes the alert to one or more PSM terminals 1102 or mobile devices 1104. According to at least one embodiment, communication between the AI device 1000 and the Alert

Router occurs via the hospital intranet, ethernet instead of a cloud based system. This aids in addressing concerns related to privacy and/or latency.

If, for example, the clinical integration layer 212 of FIG. 2 triggers an alert, then a notification is sent to a device 1104 operable by a second PSM professional. By way of example, a text message may be sent via SMS or MMS to a Provider application used by the second PSM personnel. The notification may also be sent via a cloud-hosted backend (e.g., Call Management System (CMS), DMS). According to at least one embodiment, the CMS and DMS are located on-premises (e.g., in the hospital or within the hospital network). This would improve latency and privacy if the backend systems are inside the same network as the second PSM professional.

According to at least one embodiment, a direct connection between patient-side device and PSM computers is utilized instead of a cloud-based infrastructure.

The above-described present disclosure can be implemented with computer-readable code in a computer-readable medium (e.g., non-transitory computer-readable medium) in which program has been recorded. The computer-readable medium may include all kinds of recording devices capable of storing data readable by a computer system. Examples of the computer-readable medium may include a hard disk drive (HDD), a solid state disk (SSD), a silicon disk drive (SDD), a ROM, a RAM, a CD-ROM, magnetic tapes, floppy disks, optical data storage devices, and the like and also include such a carrier-wave type implementation (for example, transmission over the Internet). Therefore, the above embodiments are to be construed in all aspects as illustrative and not restrictive. The scope of the invention should be determined by the appended claims and their legal equivalents, and not by the above description, and all changes coming within the meaning and equivalency range of the appended claims are intended to be embraced therein.

What is claimed is:

1. A method of determining a predictive index indicating a level to which a human subject is at risk of developing hypoactive delirium, the method comprising:
extracting first features of first audio content and first video content continuously capturing the human subject in a setting over a first period, the extracted first features detailing a plurality of first behavioral aspects of the human subject over the first period, wherein at least one of the plurality of first behavioral aspects is detailed at a granularity that is undetectable by a human observer;
establishing a behavioral baseline specific to the human subject based on the extracted first features;
providing the established behavioral baseline to a neural network;
extracting second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer;
providing the extracted second features to the neural network for determining the predictive index indicating the level to which the human subject is at risk of developing hypoactive delirium, based on the established behavioral baseline and the extracted second features; and
outputting an alert based on the determined predictive index being above a threshold value.

2. The method of claim 1, wherein, based on the determined predictive index being less than or equal to the threshold value, the extracted second features are provided to the neural network to train the neural network to tailor the established behavioral baseline based on the extracted second features.

3. The method of claim 2, wherein, based on the determined predictive index being less than or equal to the threshold value, the method further comprises:
extracting third features of third audio content and third video content continuously capturing the human subject in the setting over a third period subsequent to the second period, the extracted third features detailing a plurality of third behavioral aspects of the human subject over the third period, wherein at least one of the plurality of third behavioral aspects is detailed at a granularity that is undetectable by the human observer; and
providing the extracted third features to the neural network for determining the predictive index based on the tailored behavioral baseline and the extracted third features.

4. The method of claim 1, wherein a duration of the first period is in a range of 24 to 48 hours.

5. The method of claim 1, wherein the first audio content comprises windowed samples that overlap one another.

6. The method of claim 5, wherein each of the windowed samples is five seconds in length.

7. The method of claim 1, wherein the first video content comprises video content recorded by at least one day vision camera and video content recorded by at least one night vision camera.

8. The method of claim 1, wherein the plurality of second behavioral aspects of the human subject comprises at least one of hand jitter, body movement, speech activity or sleep activity of the human subject.

9. The method of claim 8,
wherein the plurality of second behavioral aspects of the human subject comprises the speech activity of the human subject, and
wherein the neural network determines the predictive index further based on speech characteristics of larger populations of human subjects.

10. The method of claim 1, wherein the behavioral baseline is established and the predictive index is determined without using data output by an electroencephalogram (EEG) machine.

11. An artificial intelligence (AI) device configured to determine a predictive index indicating a level to which a human subject is at risk of developing hypoactive delirium, the AI device comprising:
at least one transceiver; and
at least one processor configured to:
extract first features of first audio content and first video content continuously capturing the human subject in a setting over a first period, the extracted first features detailing a plurality of first behavioral aspects of the human subject over the first period, wherein at least one of the plurality of first behavioral aspects is detailed at a granularity that is undetectable by a human observer;
establish a behavioral baseline specific to the human subject based on the extracted first features;
provide the established behavioral baseline to a neural network;
extract second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer;
provide the extracted second features to the neural network for determining the predictive index indicating the level to which the human subject is at risk of developing hypoactive delirium, based on the established behavioral baseline and the extracted second features; and
output an alert based on the determined predictive index being above a threshold value.

12. The AI device of claim 11, wherein, based on the determined predictive index being less than or equal to the threshold value, the extracted second features are provided to the neural network to train the neural network to tailor the established behavioral baseline based on the extracted second features.

13. The AI device of claim 12, wherein, based on the determined predictive index being less than or equal to the threshold value, the at least one processor is further configured to:
extract third features of third audio content and third video content continuously capturing the human subject in the setting over a third period subsequent to the second period, the extracted third features detailing a plurality of third behavioral aspects of the human subject over the third period, wherein at least one of the plurality of third behavioral aspects is detailed at a granularity that is undetectable by the human observer; and
provide the extracted third features to the neural network for determining the predictive index based on the tailored behavioral baseline and the extracted third features.

14. The AI device of claim 11, wherein a duration of the first period is in a range of 24 to 48 hours.

15. The AI device of claim 11,
wherein the first audio content comprises windowed samples that overlap one another, and
wherein each of the windowed samples is five seconds in length.

16. The AI device of claim 11, wherein the first video content comprises video content recorded by at least one day vision camera and video content recorded by at least one night vision camera.

17. The AI device of claim 11, wherein the plurality of second behavioral aspects of the human subject comprises at least one of hand jitter, body movement, speech activity or sleep activity of the human subject.

18. The AI device of claim 17,
wherein the plurality of second behavioral aspects of the human subject comprises the speech activity of the human subject, and
wherein the neural network determines the predictive index further based on speech characteristics of larger populations of human subjects.

19. The AI device of claim 11, wherein the behavioral baseline is established and the predictive index is determined without using data output by an electroencephalogram (EEG) machine.

20. A non-transitory storage medium storing instructions that, when executed, cause at least one processor to perform operations, the operations comprising:
extracting first features of first audio content and first video content continuously capturing a human subject in a setting over a first period, the extracted first features detailing a plurality of first behavioral aspects of the human subject over the first period, wherein at least one of the plurality of first behavioral aspects is detailed at a granularity that is undetectable by a human observer;
establishing a behavioral baseline specific to the human subject based on the extracted first features;
providing the established behavioral baseline to a neural network;
extracting second features of second audio content and second video content continuously capturing the human subject in the setting over a second period subsequent to the first period, the extracted second features detailing a plurality of second behavioral aspects of the human subject over the second period, wherein at least one of the plurality of second behavioral aspects is detailed at a granularity that is undetectable by the human observer;
providing the extracted second features to the neural network for determining a predictive index indicating a level to which the human subject is at risk of developing hypoactive delirium, based on the established behavioral baseline and the extracted second features; and
outputting an alert based on the determined predictive index being above a threshold value.
